(19) United States
(12) Patent Application Publication
(10) Pub. No.: US 2021/0113498 A1
(43) Pub. Date: Apr. 22, 2021

(54) TOPICAL AND TRANSDERMAL DELIVERY OF AN IRON CHELATOR TO PREVENT AND TREAT CHRONIC WOUNDS

(71) Applicant: TAUTONA GROUP IP HOLDING COMPANY, L.L.C., Redwood City, CA (US)

(72) Inventor: Geoffrey C. GURTNER, Stanford, CA (US)

(73) Assignee: TAUTONA GROUP IP HOLDING COMPANY, L.L.C., Redwood City, CA (US)

(21) Appl. No.: 17/041,108

(22) PCT Filed: Mar. 27, 2019
(86) PCT No.: PCT/US2019/024405

§ 371 Date: Sep. 24, 2020

Related U.S. Application Data
(60) Provisional application No. 62/648,731, filed on Mar. 27, 2018.

(51) Int. Cl. A61K 31/16; A61K 9/107; A61K 47/32; A61K 47/38; A61K 9/70; A61P 17/02

(52) U.S. Cl. CPC A61K 31/16; A61K 9/1075; A61P 17/02; A61K 47/38; A61K 9/703; A61K 47/32

(57) ABSTRACT

A transdermal patch for the treatment of Sickle Cell Ulcers is provided. The patch can facilitate the delivery of an iron chelator, such as DFO. The DFO can be encapsulated in a reverse micelle to enhance penetration into and absorption by the dermis. The patch can be used to accelerate healing and reduce pain associated with Sickle Cell Ulcers.

### INCORPORATION BY REFERENCE

All publications and patent applications mentioned in this specification are incorporated herein by reference in their entirety to the same extent as if each individual publication or patent application was specifically and individually indicated to be incorporated by reference.

### BACKGROUND

Nonhealing chronic wounds are a challenge to the patient, the health care professional, and the health care system. They significantly impair the quality of life for millions of people and impart burden on society in terms of lost productivity and health care dollars.

Wound healing is a dynamic pathway that optimally leads to restoration of tissue integrity and function. A chronic wound results when the normal reparative process is interrupted. By understanding the biology of wound healing, the physician can optimize the tissue environment in which the wound is present. Wound healing is the result of the accumulation of processes, including coagulation, inflammation, ground substance and matrix synthesis, angiogenesis, fibroplasia, epithelialization, wound contraction, and remodeling.

In chronic wounds, the process is disrupted, and thus healing is prolonged and incomplete. A chronic wound occurs when some factor causes the disruption of the normal, controlled inflammatory phase or the cellular proliferative phase. Thus, each wound should be evaluated to determine what factors are present and how to correct the problem.

Underlying systemic disease in a patient with a wound can increase the probability that the wound will become chronic. Diabetes mellitus is one example. Wound healing is often delayed because of interruption of the inflammatory and proliferative phases. Neutrophils and macrophages cannot adequately keep the bacterial load of the wound controlled, and infection prolongs the inflammatory phase. Erythrocytes can be affected by glycosylation, leading to microvascular sludging and ischemia. Low tissue oxygen tension impairs cellular proliferation and collagen synthesis.

Common chronic skin and soft tissue wounds include diabetic foot ulcers, pressure ulcers, venous leg ulcers and sickle cell ulcers. While each of these chronic wounds has a common presentation (e.g., an open sore in the dermis and epidermis), each has a different etiology, and one would not necessarily expect a therapy effective to treat one type of chronic skin ulcer would be effective to treat other types.

Diabetic ulcers are a common cause of foot and leg amputation. In patients with type I and type II diabetes, the incidence rate of developing foot ulcers is approximately 2% per year. The pathogenesis of a diabetic foot ulcer is unique and is initiated by high levels of glycemia that build up in the skin, leading to the onset of peripheral neuropathy as the diabetes progresses. The high levels of glycemia interfere with the normal angiogenic response to hypoxia (hypoxia induction factor alpha—HIF-1α) in areas with repeated pressure insults (usually weight bearing surfaces like the bottom of the foot) making these areas susceptible to breakdown from prolonged ischemic conditions. As neuropathy progresses, the pain sensation associated with these ischemic area(s) is lost, allowing the ulcer to form unknowingly and unchecked. Inflammation and elevated levels of reactive oxygen species (ROS) accompany the ulcer formation, and a blunted leukocyte response increases the likelihood of infection.

While hyperglycemia makes diabetics susceptible to ulcers due to corrupt hypoxia response (HIF1-α), the natural decrease of HIF1-α in aged tissue, and a decrease in efficiency in hypoxia response, makes seniors naturally more susceptible to pressure ulcers. Pressure ulcers are the result of prolonged, unrelieved pressure over a bony prominence that leads to ischemia. The wounds tend to occur in older patients in whom the level HIF-1alpha is low because of upregulation of prolyl hydroxylase domain (PHD) and Factor inhibiting hypoxia-inducible factor (FIH) enzymes in patients who are unable to reposition themselves to off-load weight, such as paralyzed, unconscious, or severely debilitated persons.

Sickle cell ulcers (SCUs) are a devastating comorbidity affecting patients with sickle cell disease (SCD). SCD results from a mutation of the hemoglobin gene that generates abnormal hemoglobin and sickle-shaped erythrocytes. Their shape and lack of flexibility make sickle erythrocytes less conducive to normal blood flow, resulting in rupturing, mechanical occlusion of small blood vessels, repetitive local ischemic events and formation of SCUs. Sickle cell erythrocytes also display an abnormal level of membrane associated iron and excessive superoxide production. Furthermore, chronic hemolysis leads to an accumulation of heme in plasma and in tissues, resulting in increased reactive oxygen species (ROS) both through the Fenton reaction and from neutrophil and monocyte-derived oxidative burst. Increased ROS leads to tissue free radical defense mechanisms, yielding DNA damage, excessive inflammation and chronic ulceration. There is also microvascular occlusion in these patients resulting in excessive pain.

Patients with the disease start to manifest SCUs after 10 years of age. The pathobiology of SCUs is multifactorial, involving both local and systemic dysfunction such as vasculopathy and chronic inflammation, and it differs from the pathobiology of other chronic skin ulcers. As described in the SCU Formation flow chart of FIG. 1, sickle red blood cells (sRBCs) are more fragile than normal red blood cells (RBC's) and are subject to rupture, causing free release of hemoglobin into the blood stream. The free hemoglobin precipitates, causing it to bind the vasodilator, nitric oxide (NO). The loss of a major vasodilator facilitates further sequestration of sRBCs leading to microvascular occlusion and extreme pain. Rupturing sRBCs also cause inflammation, increased reactive oxygen species (ROS), and local ischemia with progression to ulceration. SCUs form over the medial or lateral malleoli of the lower extremity and are prone to infection and recidivism. Some SCUs may never heal, leading to pain, deformities and amputations.

There is no standardized protocol for treating SCUs. SCUs are currently treated like other chronic wounds. Surgical debridement is performed to remove dead and infected tissue in an effort to allow granulation to occur. Dressings are applied that serve to absorb excess exudate while maintaining a moist wound surface. Wound care is fairly labor-intensive, however, and requires multiple repeated clinic visits on a weekly basis. Despite these efforts, SCUs are slow to heal, if they heal at all, and they are prone to recurrence. Due to the systemic and local dysfunctions described above, granulation is inhibited at the wound site, and healing is delayed. While antibiotics may also be considered for wounds with obvious purulence, cellulitis, or osteomyelitis, there is little data to support either systemic or local antibiotic therapy for SCUs.

Iron overload commonly develops in SCD patients. Patients receive repeated blood transfusions to replace sickle erythrocytes, which can lead to free iron accumulation and organ injury, most critically in the heart and liver. Destruction of sRBCs and repeated transfusions can lead to excessive amounts of free iron in the blood. Systemically delivered deferoxamine (DFO) has been approved in the United States since 1968 for treating iron overload in SCD patients. To chelate excessive iron from their blood, SCD patients are administered DFO, either delivered subcutaneously over 8-24 hours with the use of a portable infusion pump (not to exceed 20-40 mg/kg/day), intravenously over 8-12 hours (20-40 mg/kg/day for children and 40-50 mg/kg/day for adults) or intramuscularly, not exceeding a daily dose of 1000 mg. DFO maintains a maximal affinity for ferric iron, forming an exceptionally stable hexadentate ligand, ferrioxamine, following chelation. Parenteral administration of DFO has not helped diminish the incidence, severity or persistence of SCUs, however.

DFO has also been applied transdermally to treat conditions other than SCUs. For example, U.S. Pat. No. 4,397,867 describes transdermal application of DFO to treat arthritis, but it does not describe the use of any transdermal delivery device or any particular transdermal delivery method.

U.S. Pat. No. 6,156,334 describes the use of DFO in a wound covering to promote wound healing by trapping excess iron out of the extracellular fluid in the wound. The DFO is covalently bonded to the wound covering material, however, and is not delivered into the tissue in and around the wound so that any trapped iron can be removed from the wound when the wound covering is removed. U.S. Pat. No. 6,156,334 also notes how difficult it is for DFO to penetrate into and through tissue, thereby making it an ideal “trapper molecule” for removal of iron from the wound. In these methods, the iron is only chelated from the exudate or the surface of the wound. The DFO does not penetrate below the skin.

U.S. Pat. Nos. 8,829,051 and 9,737,511 describe the administration of reactive oxygen species inhibitors (such as DFO) in a variety of forms (e.g., orally, parenterally, transdermally, subcutaneously, intravenously, intramuscularly, intraperitoneally, by intraversal instillation, intracularly, intranasally, intraarterially, and intralesionally) to treat diabetic ulcers, such as diabetic foot ulcers. These patents do not describe any transdermal delivery device or transdermal delivery method, however, nor do they address the treatment of SCUs.

US Publication No. 2010/0092546 describes compositions and methods for the treatment of chronic wounds (including pressure ulcers and diabetic ulcers, but not including SCUs) by the transdermal delivery of agents that increase activity of a hypoxia-inducible factor HIF-la potentiating agent, such as DFO, using a transdermal patch having DFO in a hydrogel or biodegradable polymer. US Publication No. 2014/0370078 describes the transdermal delivery of DFO by encapsulating the DFO in nonionic surfactants and polymers (reverse micelle encapsulation), dispersed in a release-controlling polymer matrix (e.g., ethyl cellulose) to enhance delivery into the skin. These publications describe the use of DFO, as well as other HIF-1α modulators, to increase HIF-1α activity in the wound. While these publications describe the use of DFO to treat pressure ulcers in skin of diabetic mice, they do not address the use of DFO delivered into the dermis from a delivery device applied to the skin surface to treat SCUs.

### SUMMARY

In a first aspect, a method to treat a skin ulcer caused by iron toxicity and free radical damage is provided. The method comprises contacting the ulcer and surrounding skin with intradermal patch comprising a film comprising deferoxamine (DFO) encapsulated in a reverse micelle with a non-ionic surfactant within a matrix; releasing the encapsulated DFO from the matrix over a treatment period; and penetrating the DFO into the ulcer and surrounding skin.

In some embodiments, the skin ulcer is a sickle cell ulcer. The skin ulcer can be a venous leg ulcer. In some embodiments, the patient has a blood disorder or disease making them susceptible to ulcer formation. The matrix can comprise polyvinylpyrrolidine (PVP) and ethylcellulose. In some embodiments, the film comprises DFO at a concentration of from at least about 1% and not more than about 20% as weight/weight percent of film. The film can comprise DFO at a concentration of about 1-2 mg/cm². In some embodiments, the patch comprises a length of about 60-175 mm. The patch can comprise a width of about 75-400 mm.

In another aspect, a method to treat a skin ulcer caused by iron toxicity and free radical damage is provided. The method comprises contacting the ulcer and surrounding skin with a transdermal patch comprising an iron chelator; releasing portions of the iron chelator from the transdermal patch over a treatment period; and penetrating the iron chelator into the ulcer and surrounding skin.

In some embodiments, the iron chelator comprises DFO. The iron chelator can be adapted to enhance penetration of a stratum corneum layer of the skin and/or be released in a sustained manner into the dermis. In some embodiments, the iron chelator is encapsulated in a reverse micelle. The skin ulcer can be a sickle cell ulcer. In some embodiments, the skin ulcer is a venous leg ulcer. The skin ulcer can be on a patient with blood disorder or rare disease making them susceptible to skin ulcers.

In another aspect, deferoxamine (DFO) prepared for use in local treatment of a sickle cell ulcer is provided.

The DFO can be encapsulated in a reverse micelle. In some embodiments, the DFO is contained within a transdermal patch.

In yet another aspect, deferoxamine (DFO) for use in treatment of a skin ulcer caused by iron toxicity and free radical damage is provided. The DFO is prepared for release to, and penetration into, the skin ulcer and surrounding skin.

In some embodiments, the DFO is encapsulated in a reverse micelle. The DFO can be contained within a transdermal patch. In some embodiments, the patch comprises a film comprising deferoxamine (DFO) encapsulated in a reverse micelle with a non-ionic surfactant within a matrix. The concentration of the DFO is about 1-2 mg/cm². In some embodiments, a concentration of the DFO is from at least about 1% and not more than about 20% as weight/weight percent of film. A length of the patch can be about 60-175 mm. A width of the patch can be about 75-400 mm.

In another aspect, a method to reduce pain associate with a skin ulcer caused by iron toxicity and free radical damage is provided. The method comprises contacting the ulcer and surrounding skin with intradermal patch comprising a film comprising deferoxamine (DFO) encapsulated in a reverse micelle with a non-ionic surfactant within a matrix; releasing the encapsulated DFO from the matrix over a treatment period; and penetrating the DFO into the ulcer and surrounding skin.

The skin ulcer can be a sickle cell ulcer. In some embodiments, the skin ulcer is a venous leg ulcer. In some embodiments, the skin ulcer is on a patient with blood disorder or rare disease making them susceptible to skin ulcers. The matrix can comprise polyvinylpyrrolidine (PVP) and ethylcellulose. In some embodiments, the film comprises DFO at a concentration of from at least about 1% and not more than about 20% as weight/weight percent of film. The film can comprise DFO at a concentration of about 1-2 mg/cm². In some embodiments, the patch comprises a length of about 60-175 mm. The patch can comprise a width of about 75-400 mm.

In another aspect, a method to reduce pain associated with a skin ulcer caused by iron toxicity and free radical damage is provided. The method comprises contacting the ulcer and surrounding skin with a transdermal patch comprising an iron chelator; releasing portions of the iron chelator from the transdermal patch over a treatment period; and penetrating the iron chelator into the ulcer and surrounding skin.

The iron chelator can comprise DFO. In some embodiments, the iron chelator is adapted to enhance penetration of a stratum corneum layer of the skin. The iron chelator can be adapted to be released in a sustained manner into the dermis. In some embodiments, the iron chelator is encapsulated in a reverse micelle. The skin ulcer can be a sickle cell ulcer. The skin ulcer can be a venous leg ulcer. In some embodiments, the skin ulcer is on a patient with blood disorder or rare disease making them susceptible to skin ulcers.

In another aspect, deferoxamine (DFO) prepared for use in reduction of pain associated with a sickle cell ulcer through local administration is provided.

The DFO can be encapsulated in a reverse micelle. In some embodiments, the DFO is contained within a transdermal patch.

In another aspect, deferoxamine (DFO) for use in reduction of pain associated with a skin ulcer caused by iron toxicity and free radical damage is provided. The DFO is prepared for release to, and penetration into, the skin ulcer and surrounding skin.

In some embodiments, the DFO is encapsulated in a reverse micelle. The DFO can be contained within a transdermal patch. In some embodiments, the patch comprises a film comprising deferoxamine (DFO) encapsulated in a reverse micelle with a non-ionic surfactant within a matrix. A concentration of the DFO can be about 1-2 mg/cm². In some embodiments, a concentration of the DFO is from at least about 1% and not more than about 20% as weight/weight percent of film. A length of the patch can be about 60-175 mm. A width of the patch can be about 75-400 mm.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1 is a flowchart outlining SCU formation.

FIG. 2 is a flowchart demonstrating the effect of intradermal delivery of DFO on SCU formation, as described herein.

FIG. 3A shows a representation of the generation of HbSS BERK mice.

FIGS. 3B-3D demonstrate the wound healing of mice treated with DIDP and untreated mice.

FIGS. 4A-4C demonstrate the wound healing of mice treated with DIDP and mice with DFO solution injected in their wounds daily.

FIG. 5A shows the collagen deposition in DIDP treated mice, untreated mice, and DFO-injection treated mice.

FIG. 5B shows dermal thickness in DIDP treated mice, untreated mice, and DFO-injection treated mice.

FIGS. 6A and 6B show iron presence in DIDP treated mice, untreated mice, and DFO-injection treated mice.

FIGS. 6C and 6D show the molecular composition of deferoxamine and ferroxamine.

FIGS. 7A-7F shows a schematic representation of the application of an intradermal iron chelator on an SCU.

FIG. 8 depicts a vertical Franz Diffusion Cell.

FIG. 9A shows the release of DFO from an embodiment of an intradermal delivery device.

FIG. 9B depicts relative DFO concentration using an embodiment of an intradermal delivery device and by dripping on an aqueous solution including the DFO.

FIG. 9C shows intracellular iron aggregates in the dermis.

FIG. 9D depicts dermal penetration of DFO delivered by an embodiment of an intradermal delivery device.

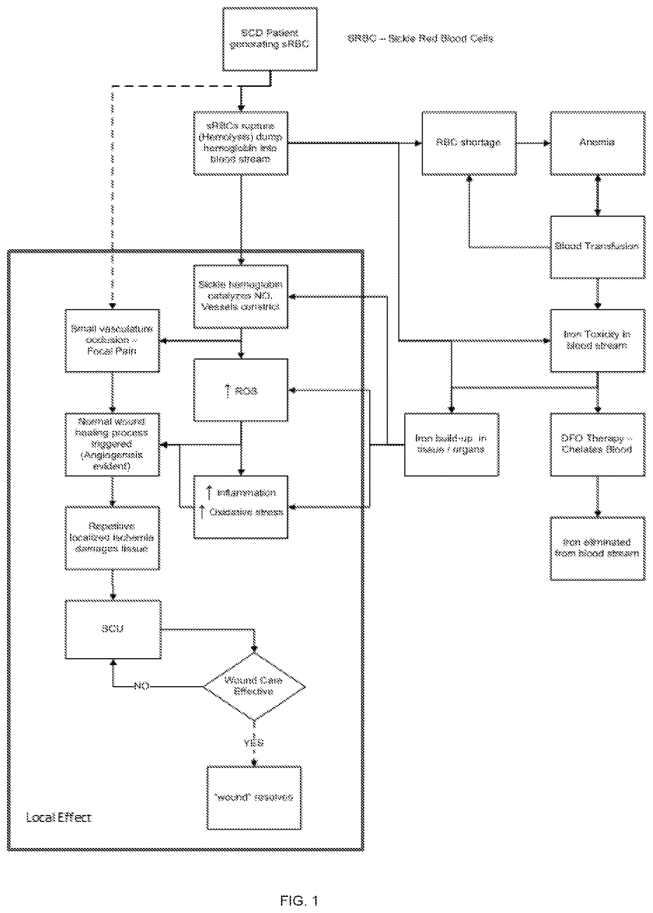

### DETAILED DESCRIPTION

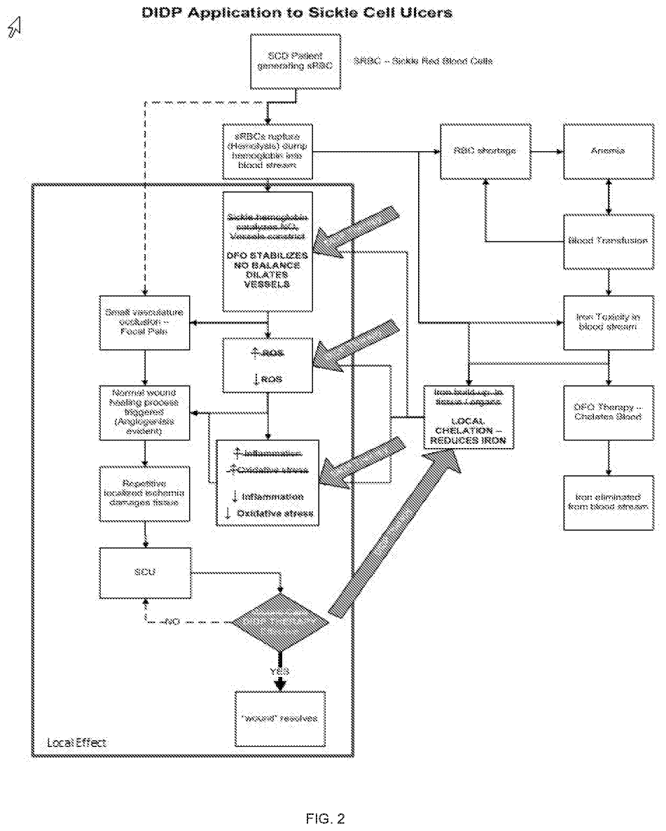

One aspect of the invention provides a method of treating an SCU by delivering an iron chelator, such as DFO, to the wound locally at a slow and sustained rate. As described below, one way to practice this invention is to deliver the iron chelator from a topical patch (e.g., a deferoxamine intradermal delivery patch or DIDP) that provides the chelator over an extended period of time and in a manner that enables the chelator to penetrate the tissue. This local delivery chelates iron around the wound, reducing free radicals created with iron and their associated oxidative stress, reducing inflammation and stabilizing vasoconstricting mechanisms that reduce blood flow. An iron chelator, such as DFO, allows the oxygen-dependent pathways to function by inhibiting hydroxylases that catalyze degradation of a transcription factor necessary for angiogenesis. Once oxygen-dependent pathways are restored, neovascular perfusion to the wound can be established, as shown in the flow chart of FIG. 2.

Another aspect of the invention provides a method of treating ulcers caused by iron toxicity and consequent free radical damage, such as sickle cell ulcers and venous leg ulcers. According to this method, an iron chelator (such as, but not limited to, DFO) is applied to the ulcer and surrounding skin topically. The DFO or other iron chelator may be prepared in a manner that enhances its ability to penetrate into the ulcer and surrounding healthy tissue and through the stratum corneum of the skin into the dermis. The DFO or other iron chelator may be applied from a transdermal or intradermal patch. The DFO or other iron chelator may be applied to the ulcer and surrounding skin over a treatment period. As described above, systemically delivered DFO is FDA-approved for treating iron overload in sickle cell patients. Typically, the drug is either delivered subcutaneously over 8-24 hours with the use of a portable infusion pump (not to exceed 20-40 mg/kg/day), intravenously over 8-12 hours (20-40 mg/kg/day for children and 40-50 mg/kg/day for adults) or intramuscularly, not exceeding a daily dose of 1000 mg (Vichinsky et al. 2006). There has been no evidence of toxicity in adult or pediatric patients when treated within these dose limits.

Systemic administration of iron chelators such as DFO is used for treating iron overload of sickle cell patients, but is not used to addressing the unique concomitant healing challenges associated with SCD such as SCUs. Parenteral administration of iron chelators, such as DFO, has also not been shown to help in addressing the unique concomitant healing challenges associated with SCD such as SCUs.

Nonetheless, it was hypothesized that targeted iron chelation of an SCU would improve healing. It was found that a more wound-targeted application of an iron chelator, such as DFO, could address these healing challenges. DFO's hydrophilic properties make direct application to hydrophobic tissue an unlikely solution. To address this challenge, a novel patch (DIDP) using hydrophobic reverse micelles containing an iron chelator, such as DFO, to penetrate the stratum corneum and release the iron chelator subdermally in a sustained manner into the healing dermis is used. It will be appreciated

The iron chelator can be encapsulated in a reverse micelle with a nonionic surfactant, which reverse micelle is stabilized by PVP in an ethyl cellulose matrix. Surfactants of interest include, without limitation, TWEEN 85® (Polyoxyethylene (20) Sorbitan Trioleate); phospholipids such as Plurol Oleique®; TRITON X-100® (Octylphenol ethylene oxide condensate); AOT (dioctyl sulfosuccinate)-TWEEN 80® (Polysorbate 80); AOT-DOLPA (dioleyl phosphoric acid); AOT-OPE4 (p,t-octylphenoxyethoxyethanol); CTAB (cetyl trimethylammonium bromide)-TRPO (mixed trialkyl phosphine oxides); lecithin; and CTAB. Conveniently, the reverse micelle structure can be generated by dissolving the film components, e.g. hydrophilic agent, PVP, ethylcellulose and surfactant in a lower alcohol, e.g. ethanol, then drying on a hydrophobic surface to form a film, which can be held in place by a wrap or adhered to a suitable backing for use in the methods of the invention.

In some embodiments, the iron chelator (e.g., DFO) concentration of the patch is about 1 mg/cm². For example, a patch with a size of about 60 mm×75 mm can be configured to deliver about 45 mg of DFO per patch. Other concentrations are also possible (e.g., about 0.5 mg/cm², about 0.5-1 mg/cm², about 0.5-1.5 mg/cm², about 1-2 mg/cm², about 2 mg/cm², greater than about 2 mg/cm², etc.). Other patch sizes are also possible (e.g., about 1-2000 mm², about 2000-4000 mm², about 3000-4000 mm², about 2500-6500 mm², about 4000-6000 mm², about 4500-6000 mm², about 5000-6000 mm², 6000-10000 mm², 10,000-30,000 mm², 30,000-60,000 mm², 40,000-60,000 mm², 50,000-60,000 mm² etc.). In some embodiments, the patch can be configured to go around a patient's ankle, because SCUs can extend completely around the ankle. For example, the patch can comprise dimensions of about 130-170 mm×330-370 mm. Other dimensions are also possible.

In some embodiments, the patch comprises a matrix comprising an iron chelator, such as DFO, in a biodegradable polymer of ethyl cellulose or ethyl cellulose and polyvinylpyrrolidone.

As noted above, US Publication No. 2014/0370078 (“the '078 application”) describes the transdermal delivery of DFO by encapsulating the DFO in nonionic surfactants and polymers for application to target the HIF-1α regulated neovascularization cascade to improve healing in chronic wounds such as pressure and diabetic ulcers. However, because the HIF-1α regulated neovascularization cascade is not an issue in SCUs, the delivery devices described in the '078 application would not necessarily be thought to be helpful when treating an SCU. The inventors of the current application found, however, that wound targeted application of DFO does increase healing of SCUs.

Additionally, in some embodiments, the intradermal administration of iron chelators, such as DFO, described in this application can be used to treat the pain associated with SCUs. Without intending to be bound by theory, it is thought that the iron chelation of the wound and subsequent iron reduction in the wound causes a reduction in pain associated with the wound. The sickle shape of sickle cells makes blood flow susceptible to mechanical interlocking as vessel diameters get naturally smaller or because of vasoconstriction. Prolonged lack of blood flow starves the area of oxygen (hypoxia) that results in a pain crisis. Transdermal application of an iron chelator will help relieve vasoconstriction and restore blood flow and reduce and/or eliminate the pain. The pain reduction can occur with or without any perceivable healing of the wound. Because pain associated with SCUs is extreme and can lead to depression, loss of work, and, in many cases, opioid addiction, the ability to reduce the pain is a significant result.

In some embodiments, the pain reduction begins to occur within a few hours of application of the intradermal patch. Other effective times are also possible (e.g., 1-6 hours, 6-12 hours, 1 day, 1-2 days, 2 days, 1-3 days, 1-5 days, 3-5 days, etc.).

As explained in further detail below, it has also been found that sustained release of DFO throughout the day provides increase chelation of free iron compared to a single bolus injection of DFO into the wound daily. In some embodiments, the patch disclosed herein can provide a sustained release of an iron chelator, such as DFO, for up to 36 hours. A study showed, that, in some embodiments, the concentration of DFO peaks at 6 hours and was maintained until 20 hours. The DFO can remain present within the dermis until around 36 hours.

It will be appreciated that the patch disclosed herein can be used on ulcers other than SCUs and venous leg ulcers. For example, patients having a blood disorder or rare disease making them susceptible to skin ulcers can also benefit from the patches disclosed herein. Such disorders or diseases can include arterial skin ulcers, neuropathic skin ulcers, malignant ulcers, pyoderma gangrenosum, cholesterol embolism, ulcers caused by calcyphylaxi, Behcet's disease, rheumatoid arthritis related (vasculitis), Raynaud's phenomenon, Raynaud's syndrome, Raynaud's disease, systemic sclerosis, scleroderma, Hansen's disease, radiation skin damage (pre/post radiation therapy), localized muscle pain, stasis dermatitis, Bazin disease, Martorells ulcer, frostbite, trophic, varicose ulcer, cutaneous vasculitis, leukocytoclastic vasculitis, cryofibrinogenemia and cryoglobulinemia, necrobiosis lipoidica diabeticorum, warfarin induced skin necrosis, non-diabetic foot ulcer, ulcers resulting from Felty syndrome, anemia, Cooley's anemia, Thrombocythaemia, Haemolytic anaemia, and Polycythaemia.

### Example 1

The proposed mechanism for healing was investigated using a sickle cell mouse study (Rodrigues et al. publication pending). In this study, transgenic sickle cell mouse model, HbSS-BERK, were used to determine the effectiveness of DIDP. These mice do not express mouse hemoglobin and carry copies of a transgene containing human α1, γ, δ and β-sickle genes on a mixed genetic background. They recapitulate human sickle cell disease including hemolysis, reticulocytosis, anemia, extensive organ damage, shortened life span and pain.

As described in more detail below, wounds in SCD mice healed significantly slower than wild-type mice (***p<0.001). DFO-TDDS (TDDS is synonymous with DIDP)-treated wounds demonstrated significantly accelerated time to closure, reduced size, and improved wound remodeling compared with untreated wounds (***p<0.001) and DFO injection treatment (*p<0.05). DFO released from the TDDS into wounds resulted in chelation of excessive dermal-free iron.

Mice. HbSS-BERK mice do not express mouse hemoglobin and carry copies of a transgene containing human α1, γ, δ and β-sickle genes on a mixed genetic background. These mice simulate human sickle cell disease including hemolysis, reticulocytosis, anemia, extensive organ damage, shortened life span and pain26-28. HbSS-BERK mice were bred and phenotyped for human sickle hemoglobin. Genotyping for the knockout and hemoglobin transgenes was performed. Control mice (wild type) on a C57/Bl6 background [#000664] were also obtained.

DFO delivery device. A transdermal delivery system was used to deliver DFO into the dermal tissue of the mice. The delivery system comprised a dry film comprising DFO at a concentration of 13.4% weight/weight % of film encapsulated in a reverse micelle with a non-ionic surfactant stabilized by polyvinylpyrrolidine (PVP) in an ethylcellulose matrix, cut into a ⅝ inch circle and covered by a silicon sheet of the same size.

Excisional wound healing. Five-month old male HbSS BERK and wild type mice were subjected to a quantitative and reproducible model of excisional wounding using an established protocol described in Galioano et al., Wound Repair Regen 12, 485-492, doi:10.1111/j.1067-1927.2004.12404.x (2004). Briefly, after induction of anesthesia and removal of hair using a shaver and depilatory cream, two 6 mm full-thickness cutaneous wounds were excised on either side of the midline of the murine dorsum using a biopsy punch. Each wound was stented with silicone rings with outer and inner diameters of 16 mm and 10 mm, and sutured in place to prevent wound contraction.

Wound treatment. HbSS BERK mice were randomized into three treatment groups: -DFO Intra-Dermal Patch, DIDP (or DFO-DIDP (DFO delivered from the transdermal delivery system described above)), DFO-injection or untreated. Following wounding, a 6 mm punch of DIDP was placed on the wound daily or 20 μl of 100 mM DFO solution was subcutaneously injected into the wound daily. The untreated group received no injection or patch. All wounds were covered with an occlusive dressing (Tegaderm, 3M, St Paul, Minn.). Digital photographs were taken every other day. Wound area was measured using Image J software (NIH).

Histology. On closure, wounds were collected and fixed in 4% paraformaldehyde overnight and embedded in paraffin. For analysis of dermal thickness, paraffin sections were stained with Trichrome (Sigma-Aldrich) and average thickness was calculated from three measurements per high-power field per wound.

Perl's Prussian Blue Stain. Abcam iron stain kit (ab150674, Cambridge, UK) was used to display iron present in tissue sections. Histological sections were deparaffinized and rehydrated. Equal volumes of potassium ferrocyanide and hydrochloric acid solution (2%) were combined to make the iron stain solution. Slides were incubated in the solution for 3 minutes and then rinsed with distilled water. Slides were then stained with abcam nuclear fast red solution for 5 minutes and then washed four separate times with distilled water. Slides were then dehydrated in 95% ethanol, followed by absolute ethanol. Blue stain directly correlates with non-chelated iron in the skin. DFO chelates iron, forming ferrioxamine, which does not react in the Perl's Prussian blue reaction.

Statistical analysis. Results are presented as mean±s.e.m. Standard data analysis was performed using a Student's t-test. Results were considered significant for *p≤0.05, **p<0.01 and ***p<0.001.

Results

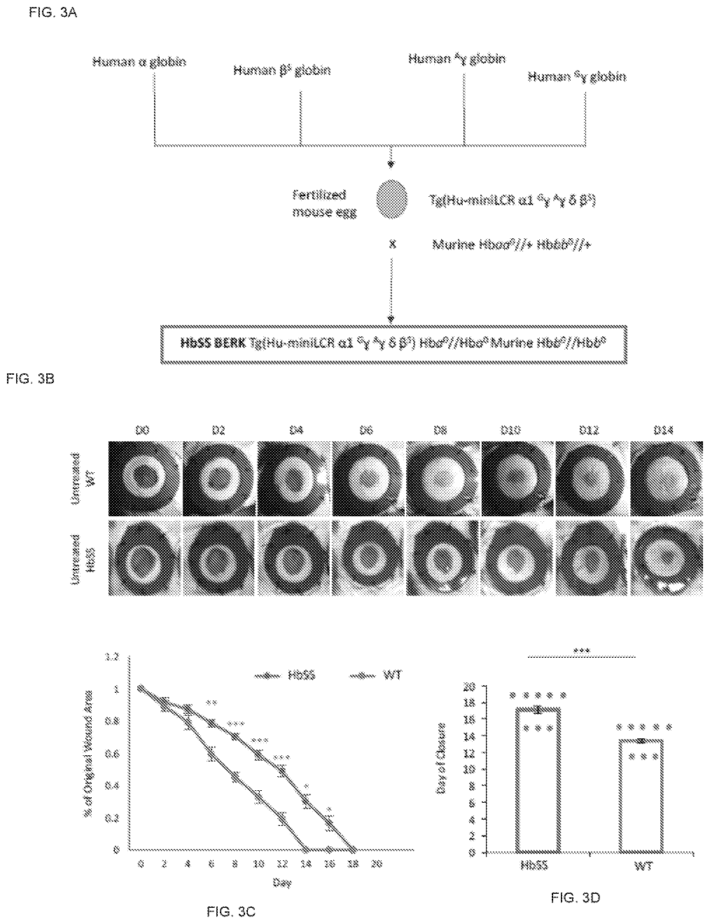

HbSS BERK mice undergo wound healing impairments compared to wild type mice. The generation of HbSS BERK mice has previously been described. Briefly, fragments of human DNA containing human α, β^{S} and γ-globin were co-injected into fertilized mouse eggs to generate a transgenic founder Tg(Hu-miniLCR α1 ^{G}γ ^{A}γ δ β^{S}) that contains human α1, γ, δ and β-sickle genes. This mouse was bred with knockout mice heterozygous for deletions of the murine α- and β-globin genes (Hbaa⁰//+Hbbb⁰//+) to generate HbSS BERK mice that were homozygous for deletion of murine α- and β-globin genes and contained the human sickle transgene. The HbSS BERK mouse is Tg(Hu-miniLCR α1 ^{G}γ ^{A}γ δ β^{S}) Hba⁰//Hba⁰ Murine Hbb⁰//Hbb⁰ (FIG. 3A).

HbSS BERK and wildtype mice were splinted following wounding to minimize contracture and to replicate human-like wound healing kinetics. Images of the excisional wounds were taken every other day, and the wound healing outcomes were assessed (FIG. 3B). HbSS BERK mice (upper line in FIG. 3C) demonstrated markedly delayed wound healing compared to wild type control mice (lower line in FIG. 3C). Differences in the wound area were statistically significant at all time-points from day 6 onwards until closure (*p<0.05, **p<0.01, ***p<0.001) (FIG. 3C). Time to complete wound closure in the HbSS BERK mice and wild type mice was 17.1429+0.4041 and 13.4+0.3055 respectively (FIG. 3D). These results indicate that the HbSS BERK mice exhibit delayed wound healing.

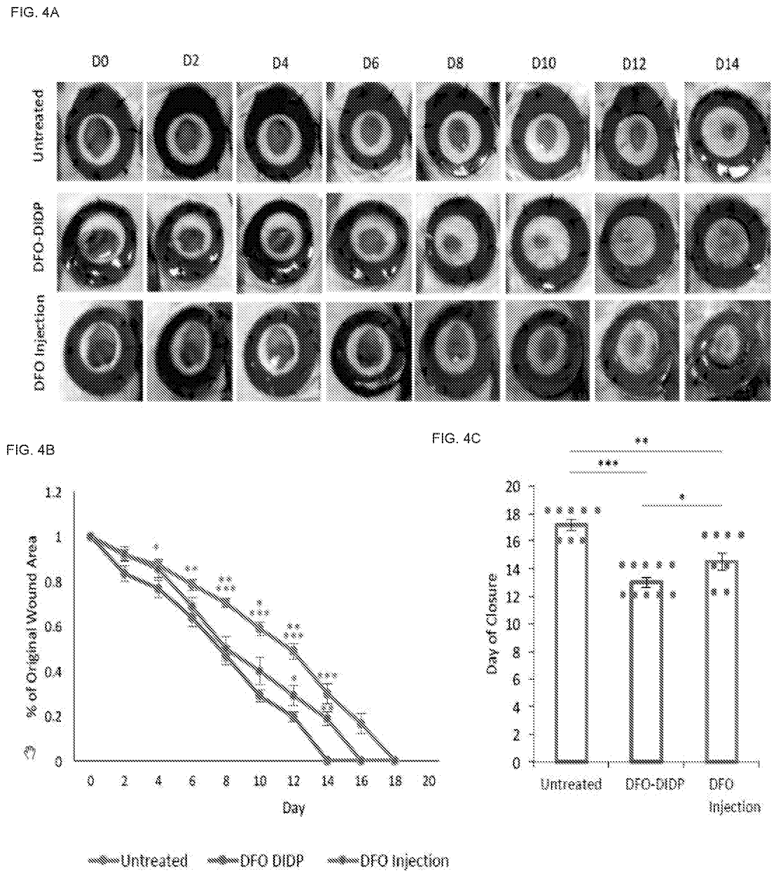

HbSS BERK wounds treated with DIDP demonstrate accelerated wound healing. Once we established that HbSS BERK mice have impaired wound healing, we treated these mice with DIDP or injected the wounds subcutaneously with DFO solution daily. Wound healing in the treated mice was compared to wounds in HbSS BERK mice that were left untreated. DIDP treated mice displayed significantly accelerated wound closure compared to both the DFO injection (*p<0.05) and the untreated group (***p<0.001) (FIG. 4A-B). In FIG. 4B, the top line shows data for the untreated mice, the middle line shows data for the DFO injected mice, and the bottom line shows data for the DIDP treated mice. Injection with DFO solution significantly reduced wound area compared to untreated controls (**p<0.01), but not as effectively as the DIDP group. Time to complete wound closure in the DIDP group, DFO injection group and untreated groups were 13+0.3660, 14.5+0.6268 and 17.1429+0.4041 respectively. (FIG. 4C). These results demonstrate that DIDP significantly accelerates wound healing and is more effective than DFO injection at treating wounds in a murine model of sickle cell ulceration.

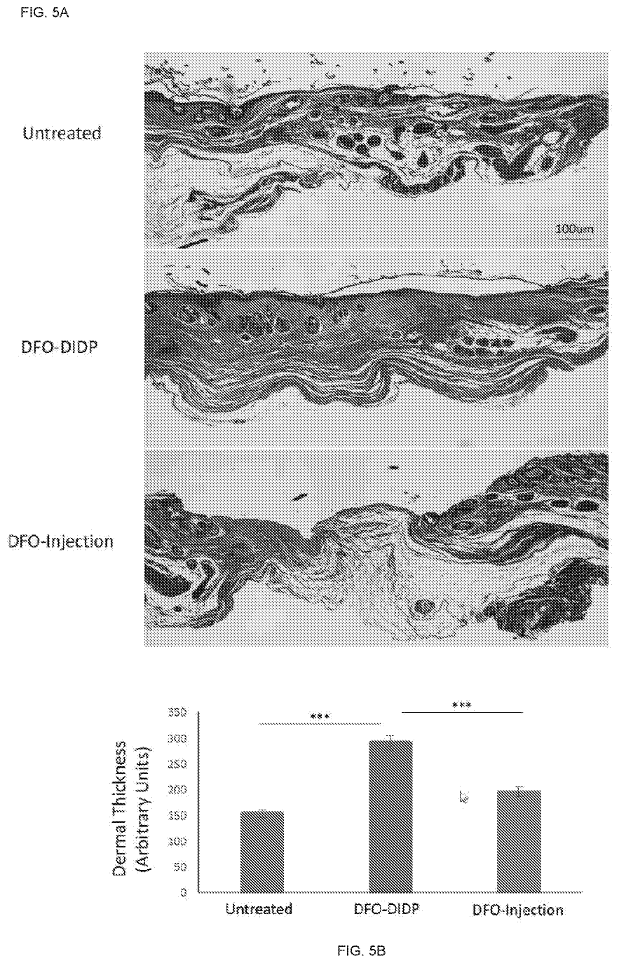

HbSS BERK wounds treated with DIDP demonstrate a thicker dermis. Histological sections of the healed wound were subjected to Trichrome analysis to determine collagen deposition in the dermis. As shown in FIG. 5A, DIDP treated wounds in the HbSS BERK mice displayed markedly greater collagen deposition in organized bundles compared to the untreated group and the DFO-injection treated mice. The width of collagen across the slides was measured to determine thickness of the dermis. Wounds treated with DIDP demonstrated significantly higher dermal thickness (*p<0.001) (FIG. 5B). Greater collagen deposition and a higher dermal thickness is desirable in the healed skin of sickle cell patients, to prevent a wound recurrence at the same site.

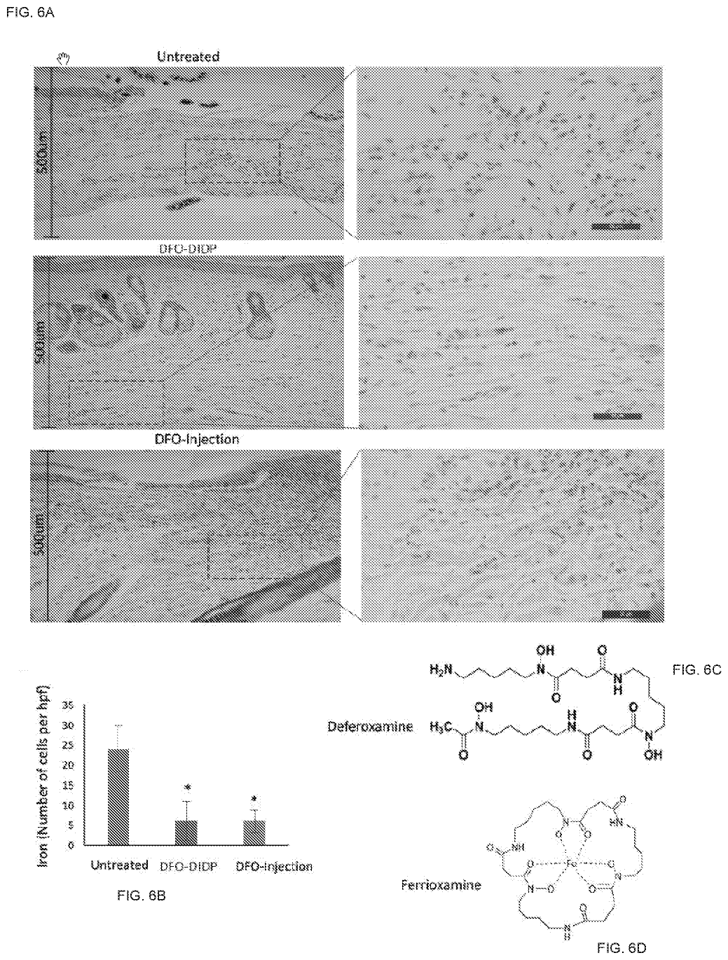

DIDP accelerates wound healing in HbSS BERK mice by chelation of free iron. Since sickle cell disease is characterized by excessive free iron leading to tissue dysfunction, histological sections of the healed wound were subjected to Perl's Prussian blue stain to determine presence of iron in untreated wounds and wounds treated with DFO. Excessive deposition of iron was observed in the untreated group, and these wound regions highly correlated with lesser dermal thickness and reduced dermal integrity (FIG. 6A-B). As shown in FIG. 6A, both DIDP and DFO-injection decreased iron in the skin as evidenced by negligible levels of Perl's Prussian blue stain (FIG. 6B). However, the DIDP treatment group demonstrated a well remodeled wound without excessive cell proliferation, uniformly bundled extracellular matrix and the return of skin appendages. DIDP treated wounds also displayed a thick dermis (over 500 μm thick), which was not observed in the untreated mice. Interestingly, the DFO-injection group showed regions of active cell proliferation and the presence of disorganized extracellular matrix, indicating the wound had not resolved healing in this treatment group. Thus, sustained release of DFO through the TDDS is more effective in healing wounds in sickle cell mice.

Next, to understand why there was reduced Prussian blue stain in the DFO-treatment and DFO-injection groups, we aimed to understand the chemical reaction by which DFO binds iron. Iron is stable when bound to six oxygen atoms. Several chelators can provide these oxygen atoms, but DFO (FIG. 6C) is known to form the strongest bonds with iron, since it is a hexadentate ligand, sharing six oxygen atoms with Fe³⁺ to form ferrioxamine (FIG. 6D). Ferrioxamine is difficult to dissociate, even in dilute solutions (10-5M) and thus withholds ferric iron from being available for the Perl's reaction. Thus, our results indicate that in the presence of DIDP, free iron in sickle cell wounds is chelated and no longer available to generate ROS and cellular damage.

Discussion

Hemoglobin S is formed by a substitution of valine for glumatic acid (GAG→GTG) at position 6 in the β-globin chain of hemoglobin A. The inheritance of two copies of this mutation (HbSS), one from each parent leads to SCD. HbSS polymerizes on deoxygenation, forming rigid sickle shaped erythrocytes. These erythrocytes impair blood flow and readily lyse leading to an accumulation of excessive free iron in the plasma and in tissues. The complications of SCD are myriad but the most common acute events during childhood are vasculopathy, vascular pain and acute chest syndrome. As the patient advances into adulthood, hemochromatosis leads to end-organ damage including chronic renal failure, stroke, avascular necrosis of bone and pulmonary hypertension. Patients with SCD also routinely experience SCUs due the buildup of iron in the skin, which affects physical function and the quality of life of the patient.

DFO is a highly effective and non-toxic iron chelator that has been routinely used to remove excessive iron from patients with hemochromatosis. Due to its short half-life, it has been administered by subcutaneous or intravenous infusion, usually over 8-12 h and for 5-7 days/week. Here, it was hypothesized that local delivery of DFO to the wound site would improve wound healing in mice with sickle cell disease by chelating excessive free iron in the wound. However, the DFO molecule is hydrophilic and relatively large, making cellular diffusion difficult.

To enable local delivery, the inventors developed a novel transdermal delivery system for an iron chelator, such as DFO (DFO-DIDP), that contains DFO encapsulated in reverse micelles. When applied topically, it allows for DFO to easily diffuse through the impermeable stratum corneum and hydrophobic membranes, with targeted delivery into the dermis. It had previously been demonstrated that DIDP improves wound healing in mice by enhancing new blood vessel formation and reducing production of reactive oxygen species (ROS).

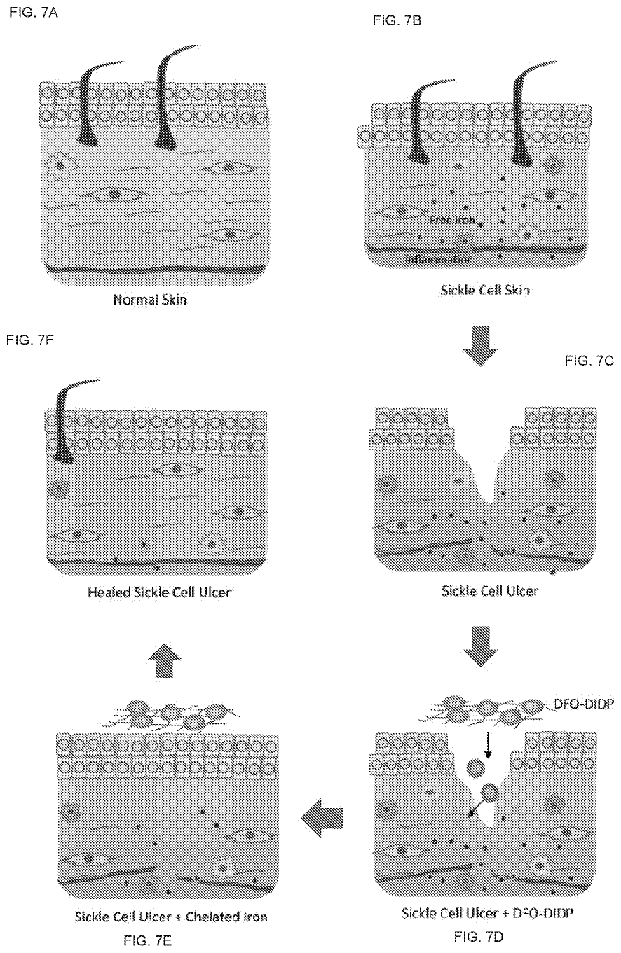

Here, a focus was on determining if DIDP could chelate iron in the skin of sickle cell mice and enhance wound healing. HbSS-BERK sickle cell mice were used for experiments. These mice contain >99% human sickle hemoglobin and display similar tissue impairments as evidenced in patients with sickle cell disease including heme-induced vaso-occlusion, decreased dermal thickness, hyperalgesia and mechanical allodynia. In experiments, HbSS BERK mice demonstrated slower wound healing compared to wild type mice. Healed wounds in untreated HbSS BERK mice displayed iron in the dermis by Perl's Prussian blue stain. Regions of high iron accumulation directly correlated with reduced dermal thickness (FIG. 7).

Daily application of DIDP on wounds significantly accelerated wound closure, increased dermal thickness and resulted in deposition of organized collagen bundles compared to both untreated wounds and wounds injected with DFO-solution daily. Importantly, DIDP treated wounds displayed negligible iron in the dermis (FIG. 7), indicating iron chelation by the formation of ferrioxamine. While the DFO-injection treated wounds also displayed negligible iron in the dermis, the wounds in this treatment group were still in the proliferative state with disorganized extracellular matrix, indicating that a single dose of DFO given once a day is not as effective as sustained release of DFO throughout the wound healing process.

Ferric iron (Fe³⁺) is much more stable under aerobic conditions in relation to ferrous iron (Fe²⁺) and is of greater significance when assessing the value of different chelators. The positive charge of ferric iron creates a particularly high charge density within the atom, thereby predisposing it to forming bonds with other atoms that possess high charge densities. Chelators such as DFO have regions of polarization, which serve as strong binding points for highly charged ferric cations. As DFO has six binding sites with ferric iron, it is labeled as a hexadentate ligand. Hexadentate ligands exhibit greater binding strength at lower concentrations than others, such as bidentate ligands, and thus, are less likely to dissociate and form hydroxyl radicals. These chemical properties make DFO a highly efficient drug to chelate excessive ferric iron in the skin. Once chelated, ferrioxamine most likely is excreted via exfoliation of epidermal cells, through sweat or might enter the blood stream and is excreted via the kidney.

The results indicate for the first time that excessive build-up of iron in the skin of HbSS sickle cell mice directly correlates with impairments in wound healing. Reducing iron locally in the skin using an FDA-approved chelator DFO significantly enhances time to wound closure, dermal thickness and wound remodeling. Sustained delivery of DFO through the DIDP system is more effective in closing wounds compared to a single bolus of DFO injected locally.

Since the DIDP system has demonstrated the ability to deliver DFO through both broken as well as intact stratum corneum, it follow that the DIDP system can be applied prophylactically to developing wounds (per-wounds) to prevent further wound development. In patients susceptible to developing iron-toxic wounds (e.g., SCUs, venous leg ulcers), the development of redness and hemosiderosis in stereotypical locations such as the medial ankle predate development of skin breakdown and wound formation.

### Example 2

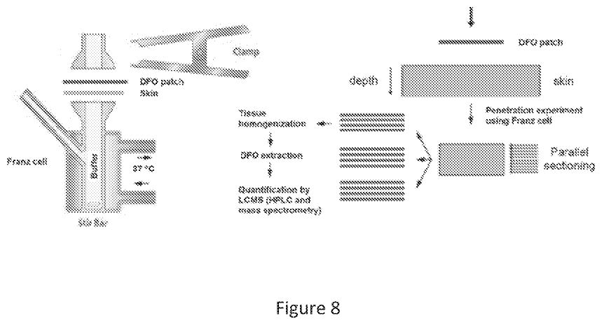

To study the capacity of an intradermal patch comprising an iron chelator, such as DFO, to deliver DFO into human dermis, a vertical Franz Diffusion Cell (FIG. 8) was used to evaluate the DIDP. Full thickness human skin samples obtained under Stanford University IRB approval were mounted between the two compartments of the diffusion cell with the stratum corneum facing the donor compartment. The DIDP was applied and isotonic phosphate buffer solution agitated with a magnetic stirrer and maintained at 37° C. by a circulating water jacket. Every hour, over the course of 15 hours, skin samples were removed, washed with phosphate buffer saline (PBS) and dried with an absorbent towel. The skin samples were frozen at −20° C. and cut with a microtome into 20 μm sections. The samples were analyzed for drug content spectrophotometrically at 560 nm (Shimadzu, Japan) and the relative DFO concentration was determined.

Skin biopsies of the fresh full thickness skin samples used in the Franz cell experiment after DIDP application were processed for histology. Intracellular DFO-iron aggregates were made visible using ferrocyanide staining via a modified Prussian blue reaction as described previously. Skin samples were incubated overnight in 7% potassium ferrocyanide dissolved in 3% hydrochloric acid. After incubation, samples were washed twice in distilled water for 5 min each and then incubated at room temperature with stable DAB containing 3% H2O2 until brown-blue color developed.

Concurrently, skin biopsies of the skin samples used in the Franz cell experiment with up to 15 hours of DIDP application were processed for mass spectrometry. Mass spectrometry for the identification of DFO molecules in human tissue section was performed as previously described using an Ultraflex I MALDI-TOF/TOF mass spectrometer (Bruker Daltonics, MA) equipped with a solid-state Smartbeam laser operating at 200 Hz.

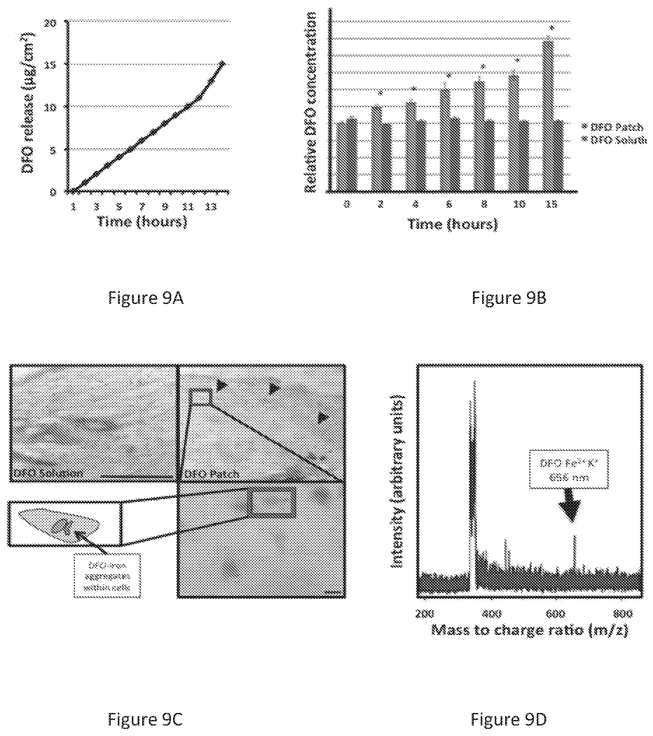

The release of DFO from the DIDP was evaluated in vitro (FIG. 9A) demonstrating that the cumulative amount of drug released by the topical patch gradually increased in a linear manner, following mixed zero-order/first-order kinetics. Having established the ability of the DIDP to provide sustained DFO delivery, its capacity for human skin permeation was evaluated. Franz Diffusion Cell experiments carried out on full thickness human cadaveric skin with DFO dripped on using an aqueous solution resulted in no permeation of the stratum corneum, while DFO eluted by the DIDP resulted in significant dermal accumulation (FIG. 9B). This was corroborated by histochemical potassium ferrocyanide staining of tissue sections from the Franz cell samples. Intracellular iron aggregates were identified in the dermis, suggesting the presence of DFO (FIG. 9C). To ensure the specificity of these findings, matrix-assisted laser desorption/ionization-time-of-flight (MALDI-TOF) mass spectrometry was conducted. MALDI-TOF analysis was able to verify the presence of DFO/iron complexes at 656 nm, indicating successful dermal penetration of DFO delivered by the DIDP (FIG. 9D). Taken together, these data demonstrate that DFO was efficiently encapsulated and delivered past the stratum corneum of human skin using a DIDP technology with an acceptable safety profile.

It is to be understood that this invention is not limited to the particular methodology, protocols, cell lines, animal species or genera, constructs, and reagents described, as such may, of course, vary. It is also to be understood that the terminology used herein is for the purpose of describing particular embodiments only, and is not intended to limit the scope of the present invention, which will be limited only by the appended claims.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for the purpose of describing and disclosing, for example, the cell lines, constructs, and methodologies that are described in the publications, which might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention.

1. A method to treat a skin ulcer caused by iron toxicity and free radical damage, the method comprising:
contacting the ulcer and surrounding skin with intradermal patch comprising a film comprising deferoxamine (DFO) encapsulated in a reverse micelle with a non-ionic surfactant within a matrix;
releasing the encapsulated DFO from the matrix over a treatment period; and
penetrating the DFO into the ulcer and surrounding skin.

2. The method of claim 1 wherein the skin ulcer is a sickle cell ulcer.

3. The method of claim 1 wherein the skin ulcer is a venous leg ulcer.

4. A method of claim 1 wherein the patient has a blood disorder or disease making them susceptible to ulcer formation.

5. The method of claim 1 wherein the matrix comprises polyvinylpyrrolidine (PVP) and ethylcellulose.

6. The method of claim 1 wherein the film comprises DFO at a concentration of from at least about 1% and not more than about 20% as weight/weight percent of film.

7. The method of claim 1, wherein the film comprises DFO at a concentration of about 1-2 mg/cm².

8. The method of claim 1, wherein the patch comprises a length of about 60-175 mm.

9. The method of claim 1, wherein the patch comprises a width of about 75-400 mm.

10. A method to treat a skin ulcer caused by iron toxicity and free radical damage, the method comprising:
contacting the ulcer and surrounding skin with a transdermal patch comprising an iron chelator;
releasing portions of the iron chelator from the transdermal patch over a treatment period; and
penetrating the iron chelator into the ulcer and surrounding skin.

11. The method of claim 10 wherein the iron chelator comprises DFO.

12. The method of claim 10 wherein the iron chelator is adapted to enhance penetration of a stratum corneum layer of the skin.

13. The method of claim 10, wherein the iron chelator is adapted to be released in a sustained manner into the dermis.

14. The method of claim 10, wherein the iron chelator is encapsulated in a reverse micelle.

15. The method of claim 10 wherein the skin ulcer is a sickle cell ulcer.

16. The method of claim 10 wherein the skin ulcer is a venous leg ulcer.

17. The method of claim 10 wherein the skin ulcer is on a patient with blood disorder or rare disease making them susceptible to skin ulcers.

18.-28. (canceled)

29. A method to reduce pain associated with a skin ulcer caused by iron toxicity and free radical damage, the method comprising:
contacting the ulcer and surrounding skin with intradermal patch comprising a film comprising deferoxamine (DFO) encapsulated in a reverse micelle with a non-ionic surfactant within a matrix;
releasing the encapsulated DFO from the matrix over a treatment period; and
penetrating the DFO into the ulcer and surrounding skin.

30. The method of claim 29 wherein the skin ulcer is a sickle cell ulcer.

31. The method of claim 29, wherein the skin ulcer is a venous leg ulcer.

32. The method of claim 29, wherein the skin ulcer is on a patient with blood disorder or rare disease making them susceptible to skin ulcers.

33. The method of claim 29 wherein the matrix comprises polyvinylpyrrolidine (PVP) and ethylcellulose.

34. The method of claim 29 wherein the film comprises DFO at a concentration of from at least about 1% and not more than about 20% as weight/weight percent of film.

35. The method of claim 29, wherein the film comprises DFO at a concentration of about 1-2 mg/cm².

36. The method of claim 29 wherein the patch comprises a length of about 60-175 mm.

37. The method of claim 29, wherein the patch comprises a width of about 75-400 mm.

38. A method to reduce pain associated with a skin ulcer caused by iron toxicity and free radical damage, the method comprising:
contacting the ulcer and surrounding skin with a transdermal patch comprising an iron chelator;
releasing portions of the iron chelator from the transdermal patch over a treatment period; and
penetrating the iron chelator into the ulcer and surrounding skin.

39. The method of claim 38 wherein the iron chelator comprises DFO.

40. The method of claim 38 wherein the iron chelator is adapted to enhance penetration of a stratum corneum layer of the skin.

41. The method of claim 38, wherein the iron chelator is adapted to be released in a sustained manner into the dermis.

42. The method of claim 38, wherein the iron chelator is encapsulated in a reverse micelle.

43. The method of claim 38, wherein the skin ulcer is a sickle cell ulcer.

44. The method of claim 38, wherein the skin ulcer is a venous leg ulcer.

45. The method of claim 38, wherein the skin ulcer is on a patient with blood disorder or rare disease making them susceptible to skin ulcers.

46.-56. (canceled)
